(19) United States
(12) Patent Application Publication
(10) Pub. No.: US 2024/0124916 A1
(43) Pub. Date: Apr. 18, 2024

(54) NANOPARTICLE WITH POLYNUCLEOTIDE BINDING SITE AND METHOD OF MAKING THEREOF

(71) Applicant: ILLUMINA CAMBRIDGE LIMITED, Cambridge (GB)

(72) Inventors: Alexandra SZEMJONOV, Cambridge (GB); Angelo LA ROSA, Biggleswade (GB); Gianluca ARTIOLI, Cambridge (GB); Xavier VON HATTEN, Cambridge (GB); Alexandre RICHEZ, Cambridge (GB)

(73) Assignee: ILLUMINA CAMBRIDGE LIMITED, Cambridge (GB)

(21) Appl. No.: 18/467,770

(22) Filed: Sep. 15, 2023

Related U.S. Application Data
(60) Provisional application No. 63/375,965, filed on Sep. 16, 2022.

(51) Int. Cl. C12Q 1/6806; B82Y 5/00; B82Y 40/00; C08L 33/02; C08L 33/26

(52) U.S. Cl. CPC C12Q 1/6806; B82Y 5/00; B82Y 40/00; C08L 33/02; C08L 33/26

(57) ABSTRACT

The present disclosure relates to a nanoparticle including a first layer including a first polymer and a first plurality of accessory oligonucleotides, a second layer including a second polymer and a single template site for bonding a template polynucleotide, and a third layer including a third polymer and a second plurality of accessory oligonucleotides. Also described herein is a method of making said nanoparticle, including “dip-coating,” e.g., successively dipping a surface with wettable nanodomains in different polymer solutions. Further described herein is a method of making the nanoparticles by forming them in nanowells and subsequently releasing them from the nanowells. Also described herein is a method of attaching the nanoparticle to a substrate and amplifying the template polynucleotide using a polymerase.

This application claims benefit of U.S. Provisional Patent Application Ser. No. 63/375,965, filed Sep. 16, 2022, which is hereby incorporated by reference in its entirety.

### FIELD

The present disclosure relates generally to multi-phasic nanoparticles having three layers of polymers and each layer including a plurality of accessory oligonucleotides or a single template site for bonding a template polynucleotide. Also described herein are methods of making such nanoparticles and methods of attaching such nanoparticles to a substrate.

### BACKGROUND

Many sequencing platforms use “sequencing by synthesis” (SBS) technology and fluorescence-based methods for detection. In some examples, template polynucleotides are attached to a surface of a substrate in a process known as seeding. Multiple copies of the template polynucleotide may be synthesized in proximity to the originally “seeded” template in a process called clustering. Subsequently, nascent copies of the clustered polynucleotides are synthesized under conditions in which they emit a signal identifying each nucleotide as it is attached to the nascent strand. Clustering of a plurality of copies of the seeded template polynucleotide in proximity to where it was initially seeded results in amplification of signal generated during the visualizable polymerization, improving detection.

Seeding and clustering work well when template polynucleotides from a library with sequences that differ from each other seed on, or attach to, positions of the surface sufficiently distal from each other such that clustering results in spatially distinct clusters of copied polynucleotides each resulting from the seeding of a single template polynucleotide, a condition generally referred to as monoclonality. If two different template polynucleotides seed too closely together on a surface of a substrate, clustering may result in spatially adjoined or comingled populations of copied polynucleotides, a condition generally referred to as polyclonality, which may result in an imaging system used in an SBS process being unable to distinguish them as separate clusters. It may be more difficult, time consuming, expensive, and less efficient, and require more complicated data analytics to obtain unambiguous sequence information from a polyclonal cluster if present. Furthermore, in simultaneous paired end reads (SPEAR) sequencing, it may be important to ensure sufficient spatial separation of read 1 and read 2 primers to avoid polyclonality.

The present disclosure is directed to overcoming these and other deficiencies in the art.

### SUMMARY

The present disclosure offers advantages, benefits, and other alternatives over known compositions and methods, by providing nanoparticles, and methods of making thereof, that ensure monoclonality during clustering prior to sequencing.

In an aspect, a nanoparticle includes a first layer including a first polymer and a first plurality of accessory sites; a second layer including a second polymer including a single template site for bonding a template polynucleotide; and a third layer including a third polymer and a second plurality of accessory sites; wherein the second layer is between the first layer and the third layer.

In an implementation, first polymer is a hydrophilic polymer. In another implementation, the hydrophilic polymer is selected from a natural polyacrylamide, a polyethylene imine, a polypeptide, a polysaccharide, a polyvinyl alcohol, a poly acrylic acid, a poly allylamine, a poly-styrene sulfonate, or a poly-oxazoline. In yet another implementation, the third polymer is a lipophilic polymer. In still another implementation, the lipophilic polymer is selected from an isopropylacrylamide, an acrylic, an epoxy, a polyethylene, a polystyrene, a polyvinyl a polymethyl sulfonate, a polyurethane, and a fluorinated polymer. In a further implementation, the first polymer and the third polymer are each independently chosen from a poly(vinylidene fluoride), a polystyrene, an epoxy polymer, a (meth)acrylate polymer, a polydimethylsiloxane, an SiO₂-containing polymer, a poly(lactic-co-glycolic acid) polymer, a perfluorinated polymer, an azapa-co-acrylamide polymer (PAZNAM), a poly(N-(5-azidoacetamidylpentyl)acrylamide-co-acrylamide) (PAZAM) polymer, a poly(o-nitrobenzyl-masked acrylamide-co-acrylamide) copolymer, a poly(benzopyrone-masked acrylamide-co-acrylamide) copolymer, a poly(aminotriazole-acrylamide-co-acrylamide) copolymer, a poly(thiotriazole-acrylamide-co-acrylamide) copolymer, a poly(alkenyltriazole-acrylamide-co-acrylamide) copolymer, and a thiol/ene cross-linkable monomer mix.

In yet a further implementation, the first polymer is polyacrylamide and the third polymer is isopropyl acrylamide. In still a further implementation, the second polymer is a copolymer of the first polymer and the third polymer. In an implementation, the second polymer is a hydrophilic polymer or a lipophilic polymer. In another implementation, the second polymer is methacylate.

In yet another implementation, the nanoparticle further including one or both of a first plurality of accessory oligonucleotides attached to the first plurality of accessory sites and a second plurality of accessory oligonucleotides attached to the second plurality of accessory sites. In still another implementation, one or both of the first plurality of accessory oligonucleotides and the second plurality of accessory oligonucleotides include one or both of forward primers and reverse primers, wherein sequences of the forward primers and sequences of the reverse primers permit amplifying the template polynucleotide by a polymerase. In a further implementation, the forward primers of the first plurality of accessory oligonucleotides and reverse primers of the second plurality of accessory oligonucleotides, or reverse primers of the first plurality of accessory oligonucleotides and forward primers of the second plurality of accessory oligonucleotides, are cleavable, and other primers of the first and second pluralities of accessory oligonucleotides are uncleavable.

In a further implementation, the single template site includes a type of attachment site for a template anchor oligonucleotide or the template anchor oligonucleotide.

In an aspect, a method includes attaching the nanoparticle of any one of claims 1 through 14 to a substrate and amplifying the template polynucleotide using a polymerase. In an implementation, the substrate includes a nanowell. In another implementation, the substrate is silanized TiO₂ or fluoro-silane TiO₂. In yet another implementation, attaching includes hybridizing a forward primer or a reverse primer to an oligonucleotide attached to the substrate.

In an aspect, a method of making a nanoparticle includes coating wettable nanodomains of a substrate surface with a solubilizable polymer, wherein the wettable nanodomains are separated by non-wettable interstices; coating the solubilizable polymer with a first polymer, wherein the first polymer includes a first plurality of accessory sites; coating the first polymer with a second polymer, wherein the second polymer includes a single template site; coating the second polymer with a third polymer, wherein the third polymer includes a second plurality of accessory sites; and solubilizing the solubilizable polymer to release the nanoparticle.

In an implementation, one or both of: (i) the first plurality of accessory sites includes a first plurality of accessory oligonucleotides, and (ii) the second plurality of accessory sites includes a second plurality of accessory oligonucleotides. In another implementation, one or both of: (i) the first plurality of accessory sites includes a first type of attachment site for a first plurality of accessory oligonucleotides but not for a second plurality of accessory oligonucleotides, and (ii) the second plurality of accessory sites includes a second type of attachment site for the second plurality of accessory oligonucleotides but not for the first plurality of accessory oligonucleotides.

In yet another implementation, the method further includes, after the solubilizing, one or more of attaching the first plurality of accessory oligonucleotides to the first plurality of accessory sites and attaching the second plurality of accessory oligonucleotides to the second plurality of accessory sites. In still another implementation, the single template site includes: a type of attachment site for a template anchor oligonucleotide and the method further includes attaching the template anchor oligonucleotide to the single template site, or; the template anchor oligonucleotide.

In a further implementation, wherein the solubilizable polymer is a sulfonate, a sugar, or a phenol. In yet a further implementation, the solubilizable polymer is poly(sodium 4-styrenesulfonate) or poly-4-vinylphenol.

In an aspect, a method includes forming a nanoparticle in a nanowell, wherein forming includes: polymerizing a first polymer, wherein the first polymer includes a first plurality of accessory sites; polymerizing a second polymer on the first polymer, wherein the second polymer includes a single template site; and polymerizing a third polymer on the second polymer, wherein the third polymer includes a second plurality of accessory sites.

In an implementation, one or both of: (i) the first plurality of accessory sites includes a first plurality of accessory oligonucleotides, and (ii) the second plurality of accessory sites includes a second plurality of accessory oligonucleotides.

In another implementation, one or both of: (i) the first plurality of accessory sites includes a first type of attachment site for a first plurality of accessory oligonucleotides but not for a second plurality of accessory oligonucleotides, and (ii) the second plurality of accessory sites includes a second type of attachment site for the second plurality of accessory oligonucleotides but not for the first plurality of accessory oligonucleotides.

In yet another implementation, the single template site includes: a type of attachment site for a template anchor oligonucleotide and the method further includes attaching the template anchor oligonucleotide to the single template site, or; the template anchor oligonucleotide.

In still another implementation, the method further includes releasing the nanoparticles from the nanowells, wherein releasing includes mechanically releasing the nanoparticles.

In an implementation, the method further includes polymerizing a solubilizable polymer before the polymerizing a first polymer. In another implementation, the method further includes releasing the nanoparticles from the nanowells, wherein releasing includes solubilizing the solubilizable polymer. In yet another implementation, the solubilizable polymer is a sulfonate, a sugar, or a phenol. In still another implementation, the solubilizable polymer is poly(sodium 4-styrenesulfonate) or poly-4-vinylphenol.

In an implementation, the first polymer is a hydrophilic polymer. In another implementation, the hydrophilic polymer is selected from a natural polyacrylamide, a polyethylene imine, a polypeptide, a polysaccharide, a polyvinyl alcohol, a poly acrylic acid, a poly allylamine, a poly-styrene sulfonate, or a poly-oxazoline. In yet another implementation, the second polymer is a copolymer of the first polymer and the third polymer. In still another implementation, the second polymer is a hydrophilic polymer or a lipophilic polymer. In a further implementation, the second polymer includes a methacylate. In yet a further implementation, the third polymer is a lipophilic polymer. In still a further implementation, the lipophilic polymer is selected from an isopropylacrylamide, an acrylic, an epoxy, a polyethylene, a polystyrene, a polyvinyl a polymethyl sulfonate, a polyurethane, and a fluorinated polymer.

In an implementation, the first polymer and the third polymer are each independently chosen from a poly(vinylidene fluoride), a polystyrene, an epoxy polymer, a (meth)acrylate polymer, a polydimethylsiloxane, an SiO₂-containing polymer, a poly(lactic-co-glycolic acid) polymer, a perfluorinated polymer, an azapa-co-acrylamide polymer (PAZNAM), a poly(N-(5-azidoacetamidylpentyl)acrylamide-co-acrylamide) (PAZAM) polymer, a poly(o-nitrobenzyl-masked acrylamide-co-acrylamide) copolymer, a poly(benzopyrone-masked acrylamide-co-acrylamide) copolymer, a poly(aminotriazole-acrylamide-co-acrylamide) copolymer, poly(thiotriazole-acrylamide-co-acrylamide) copolymer, a poly(alkenyltriazole-acrylamide-co-acrylamide) copolymer, and a thiol/ene cross-linkable monomer mix.

In another implementation, the method further includes attaching a single template polynucleotide to the single template site on the second polymer. In yet another implementation, the first plurality of accessory oligonucleotides and the second plurality of accessory oligonucleotides include forward primers and reverse primers, respectively, or reverse primers and forward primers, respectively, wherein sequences of the forward primers and sequences of the reverse primers permit amplifying the template polynucleotide by a polymerase.

In an aspect, a method of forming a nanoparticle includes coating wettable nanodomains of a substrate with a solubilizable polymer, wherein the wettable nanodomains are separated by non-wettable interstices; coating the solubilizable polymer with a first polymer including a first plurality of accessory sites; coating the first polymer with a second polymer including a single template site for bonding a template polynucleotide; coating the second polymer with a third polymer including a second plurality of accessory sites; attaching a first plurality of accessory oligonucleotides to the first plurality of accessory sites; attaching a second plurality of accessory oligonucleotides to the second plurality of accessory sites; and solubilizing the solubilizable polymer.

In an implementation, attaching a first plurality of accessory oligonucleotides to the first plurality of accessory sites occurs: before coating the solubilizable polymer with the first polymer; after coating the solubilizable polymer with the first polymer and before the solubilizing; or after the solubilizing. In another implementation, attaching a second plurality of accessory oligonucleotides to the second plurality of accessory sites occurs: before coating the second polymer with the third polymer; after coating the second polymer with the third polymer and before the solubilizing; or after the solubilizing.

In yet another implementation, the solubilizable polymer is a sulfonate, a sugar, or a phenol. In still another implementation, the solubilizable polymer is poly(sodium 4-styrenesulfonate) or poly-4-vinylphenol

In an implementation, the first polymer is a hydrophilic polymer. In another implementation, the hydrophilic polymer is selected from a natural polyacrylamide, a polyethylene imine, a polypeptide, a polysaccharide, a polyvinyl alcohol, a poly acrylic acid, a poly allylamine, a poly-styrene sulfonate, or a poly-oxazoline. In yet another implementation, the second polymer is a copolymer of the first polymer and the third polymer. In still another implementation, the second polymer is a hydrophilic polymer or a lipophilic polymer. In a further implementation, the second polymer includes a methacylate. In yet a further implementation, the third polymer is a lipophilic polymer. In still a further implementation, the lipophilic polymer is selected from an isopropylacrylamide, an acrylic, an epoxy, a polyethylene, a polystyrene, a polyvinyl a polymethyl sulfonate, a polyurethane, and a fluorinated polymer.

In an implementation, the first polymer and the third polymer are each independently chosen from a poly(vinylidene fluoride), a polystyrene, an epoxy polymer, a (meth)acrylate polymer, a polydimethylsiloxane, an SiO₂-containing polymer, a poly(lactic-co-glycolic acid) polymer, a perfluorinated polymer, an azapa-co-acrylamide polymer (PAZNAM), a poly(N-(5-azidoacetamidylpentyl)acrylamide-co-acrylamide) (PAZAM) polymer, a poly(o-nitrobenzyl-masked acrylamide-co-acrylamide) copolymer, a poly(benzopyrone-masked acrylamide-co-acrylamide) copolymer, a poly(aminotriazole-acrylamide-co-acrylamide) copolymer, poly(thiotriazole-acrylamide-co-acrylamide) copolymer, a poly(alkenyltriazole-acrylamide-co-acrylamide) copolymer, and a thiol/ene cross-linkable monomer mix.

In another implementation, the method further includes attaching a single template polynucleotide to the single template site on the second polymer. In another implementation, the first plurality of accessory oligonucleotides and the second plurality of accessory oligonucleotides include forward primers and reverse primers, respectively, or reverse primers and forward primers, respectively, wherein sequences of the forward primers and sequences of the reverse primers permit amplifying the template polynucleotide by a polymerase.

### BRIEF DESCRIPTION OF THE DRAWINGS

FIGS. 1A and 1B show a scheme showing a method of making nanoparticles for use in SBS systems;

FIG. 2 depicts a flow diagram of a method of attaching the nanoparticle disclosed herein to a substrate to amplify the template polynucleotide;

FIG. 3 depicts a flow diagram of a method of making the nanoparticle disclosed herein on the surface of a substrate;

FIG. 4 is depicts a flow diagram of a method of making the nanoparticle disclosed herein in a nanowell;

FIG. 5 depicts a flow diagram of a method of making the nanoparticle disclosed herein in a nanowell including a solubilizable layer;

FIG. 6 illustrates the reconfiguration of nanoparticles by exposure to heat; and

FIG. 7 is a scheme showing the overall workflow of making nanoparticles as disclosed herein using lithographical templates.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits and advantages described herein.

### DETAILED DESCRIPTION

An aspect of the present disclosure relates to a nanoparticle, wherein the nanoparticle includes a first layer including a first polymer and a first plurality of accessory oligonucleotides, a second layer including a second polymer including a single template site for bonding a template polynucleotide, and a third layer including a third polymer and a second plurality of accessory oligonucleotides, wherein the second layer is between the first layer and the third layer.

In an implementation, the first polymer and the third polymer are each independently chosen from a poly(vinylidene fluoride), a polystyrene, an epoxy polymer, a (meth)acrylate polymer, a polydimethylsiloxane, an SiO₂-containing polymer, a poly(lactic-co-glycolic acid) polymer, a perfluorinated polymer, an azapa-co-acrylamide polymer (PAZNAM), a poly(N-(5-azidoacetamidylpentyl)acrylamide-co-acrylamide) (PAZAM) polymer, a poly(o-nitrobenzyl-masked acrylamide-co-acrylamide) copolymer, a poly(benzopyrone-masked acrylamide-co-acrylamide) copolymer, a poly(aminotriazole-acrylamide-co-acrylamide) copolymer, a poly(thiotriazole-acrylamide-co-acrylamide) copolymer, a poly(alkenyltriazole-acrylamide-co-acrylamide) copolymer, and a thiol/ene cross-linkable monomer mix.

In another implementation, the first polymer may be a hydrophilic polymer. Non-limiting examples of hydrophilic polymers include polyethylene imine, polypeptides, polysaccharides, polyvinyl alcohol, poly acrylic acid, poly allylamine, poly-styrene sulfonate, poly-oxazoline, and the like. In yet another implementation, the first polymer is a natural polyacrylamide.

In another implementation, the third polymer may be a lipophilic polymer. Non-limiting examples of a lipophilic polymer include an acrylic, an epoxy, polyethylene, polystyrene, polyvinyl, polymethyl sulfonate, polyurethane, a fluorinated polymer, and the like. In a non-limiting example, the first and third polymers may be polyacrylamide and isopropyl acrylamide, respectively.

In another implementation, a first plurality of accessory oligonucleotides may be covalently bonded to a first polymer and/or a second plurality of accessory oligonucleotides may be covalently bonded to a second polymer. In yet another implementation, the covalent bonding may be selected from amine-NHS ester bonding sites, amine-imidoester bonding sites, amine-pentofluorophenyl ester bonding sites, amine-hydroxymethyl phosphine bonding sites, carboxyl-carbodiimide bonding sites, thiol-maleimide bonding sites, thiol-haloacetyl bonding sites, thiol-pyridyl disulfide bonding sites, thiol-thiosulfonate bonding sites, thiol-vinyl sulfone bonding sites, aldehyde-hydrazide bonding sites, aldehyde-alkoxyamine bonding sites, hydroxy-isocyanate bonding sites, azide-alkyne bonding sites, azide-phosphine bonding sites, transcyclooctene-tetrazine bonding sites, norbornene-tetrazine bonding sites, azide-cyclooctyne bonding sites, azide-norbornene bonding sites, oxime bonding sites, and any combination of two or more of the foregoing.

In a further implementation, the first plurality of accessory oligonucleotides may be noncovalently bonded to the first polymer and/or the second plurality of accessory oligonucleotides may be noncovalently bonded to the second polymer. In yet a further implementation, the non-covalent bonding includes a hybridized bond including noncovalent peptides selected from one or both of coiled-coil and avidin-biotin bonding.

In an implementation, the first plurality of accessory oligonucleotides and the second plurality of accessory oligonucleotides include forward primers and reverse primers, respectively, or reverse primers and forward primers, respectively, wherein sequences of the forward primers and sequences of the reverse primers permit amplifying the template polynucleotide by a polymerase.

In an implementation, the second polymer may be a hydrophilic polymer or a lipophilic polymer. Non-limiting examples of hydrophilic polymers include polyethylene imine, polypeptides, polysaccharides, polyvinyl alcohol, poly acrylic acid, poly allylamine, poly-styrene sulfonate, poly-oxazoline, and the like. Non-limiting examples of lipophilic polymers include an acrylic, an epoxy, polyethylene, polystyrene, polyvinyl, polymethyl sulfonate, polyurethane, fluorinated polymers, and the like. In another implementation, the second polymer may include methacylate. In yet another implementation, the second polymer may include a methacylate having a small molecular weight (Mw). In still another implementation, the methacylate may have a Mw of from about 0 kDa to about 60 kDa, including any and all subranges therein, for example, of from about 1 to about 50 kDa, of from about 1 to about 40 kDa, of from about 1 to about 30 kDa, of from about 1 to about 20 kDa, of from about 1 to about 10 kDa, of from about 10 to about 50 kDa, of from about 20 to about 50 kDa, of from about 30 to about 50 kDa, of from about 40 to about 50 kDa, of from about 50 to about 60 kDa, and the like. Molecular weight (Mw) refers to a “weight average” and may be determined by size-exclusion chromatography.

In yet another implementation, the single template polynucleotide bonding site may be of a chemistry or structure/moiety different from how the first plurality of accessory oligonucleotides or second plurality of oligonucleotides, may be attached to the first polymer or third polymer, respectively. In still another implementation, the chemistry or structure of how the first plurality of accessory oligonucleotides or the second plurality of accessory oligonucleotides attach to the first polymer or third polymer, respectively, may be different from and incompatible with the chemistry or structure of how the single template polynucleotide bonds to the single template polynucleotide bonding site of the second polymer. In another implementation, the chemistry or structure of how the first plurality of accessory oligonucleotides attach to the first polymer may be different from and incompatible with the chemistry or structure of how the second plurality of accessory oligonucleotides attach to the third polymer. In yet another implementation, the chemistry or structure of the single template polynucleotide bonding site may be different from how the first plurality of accessory oligonucleotides or second plurality of oligonucleotides, may be attached to the first polymer or third polymer, respectively, and the chemistry or structure of how the first plurality of accessory oligonucleotides attach to the first polymer may be different from and incompatible with the chemistry or structure of how the second plurality of accessory oligonucleotides attach to the third polymer. In other words, three different chemistries or structures may be chosen, a different one for each layer, such that each layer has only one chemistry or structure appropriate for attachment of one type of template or oligonucleotide, and there is no cross-attachment between layers. Examples of chemistries or structures that may be used to form a nanoparticle with orthogonal binding on the three faces is described in greater detail below.

A template polynucleotide may be a polynucleotide obtained from a sample, such as a polydeoxyribonucleic acid isolated from a sample, or a cDNA molecule copied from a mRNA molecule that was obtained from a sample. An SBS process may be performed, for example, to determine a nucleotide sequence of a template polynucleotide, or to identify one or more polymorphisms or alterations in genetic sequence of a template polynucleotide in comparison to a reference sequence. A library may be prepared from one or more samples, the library including a plurality of template polynucleotides obtained from the one or more samples. Template polynucleotides may be obtained by obtaining polynucleotide sequences that are portions of sequences that were present in the sample or copied from the sample. By sequencing a plurality of template polynucleotides in an SBS process, sequence, genotype, or other sequence-related information may be determined as to the template polynucleotides and, when sequence information about a plurality of template polynucleotides in a library may be collected and analyzed, about the sample from which the library was obtained.

A template polynucleotide may be of any given length suitable for obtaining sequencing information in an SBS process. For example, a template polynucleotide may be about 50 nucleotides in length, about 75 nucleotides in length, about 100 nucleotides in length, about 125 nucleotides in length, about 150 nucleotides in length, about 175 nucleotides in length, about 200 nucleotides in length nucleotides in length, about 225 nucleotides in length, about 250 nucleotides in length, about 275 nucleotides in length, about 300 nucleotides in length, about 325 nucleotides in length, about 350 nucleotides in length, about 375 nucleotides in length, about 400 nucleotides in length, about 425 nucleotides in length, about 450 nucleotides in length, about 475 nucleotides in length, about 500 nucleotides in length, about 525 nucleotides in length, about 550 nucleotides in length, about 575 nucleotides in length, about 600 nucleotides in length, about 625 nucleotides in length, about 650 nucleotides in length, about 675 nucleotides in length, about 700 nucleotides in length, about 725 nucleotides in length, about 750 nucleotides in length, about 775 nucleotides in length, about 800 nucleotides in length, about 825 nucleotides in length, about 850 nucleotides in length, about 875 nucleotides in length, about 900 nucleotides in length, about 925 nucleotides in length, about 950 nucleotides in length, about 975 nucleotides in length, about 1,000 nucleotides in length, about 1,025 nucleotides in length, about 1,050 nucleotides in length, about 1,075 nucleotides in length, about 1,100 nucleotides in length, about, 1,125 nucleotides in length, about 1,150 nucleotides in length, about 1,175 nucleotides in length, about 1,200 nucleotides in length, about 1,225 nucleotides in length, about 1,250 nucleotides in length, about 1,275 nucleotides in length, about 1,300 nucleotides in length, about 1,325 nucleotides in length, about 1,350 nucleotides in length, about 1,375 nucleotides in length, about 1,400 nucleotides in length, about 1,425 nucleotides in length, about 1,450 nucleotides in length, about 1,475 nucleotides in length, about 1,500 nucleotides in length, or longer.

A template polynucleotide may be processed as part of a process of obtaining a template polynucleotide from sample. Part of processing may include adding polynucleotide sequences, such as to the 5-prime, 3-prime, or both ends of the template to assist in subsequence SBS processing. As further disclosed herein, a template polynucleotide may further be modified by adding features that promote or permit forming a bond with a site on a nanoparticle. For example, the single template site of the second polymer may be an oligo attached to the second polymer, wherein the oligo is complementary to a portion of a template polynucleotide. In an implementation, template polynucleotides can have sequences added during processing, including a sequence that can hybridize to an oligonucleotide attached to the second polymer.

Attachment of a single template polynucleotide or accessory oligonucleotide to their respective polymers may be accomplished by inclusion of moieties or structures on the template polynucleotide or accessory oligonucleotide that are complementary to moieties or structures on their respective polymers, meaning they are configured to bind to one another, covalently or noncovalently, to form an attachment therebetween. Cross-reactivity between a moiety or structure attached to a template polynucleotide and a moiety or structure of the first or second polymer should be avoided to prevent attachment of more than one template polynucleotide to the nanoparticle. Further, cross-reactivity between a moiety or structure attached to an accessory oligonucleotide and a moiety or structure of the second polymer should be avoided to prevent occupation of the single template polynucleotide bonding site by an accessory oligonucleotide. In other words, attachment of each of a first plurality of oligonucleotides, a second plurality of oligonucleotides, and a single template polynucleotide to a first polymer, a third polymer, and a second polymer, respectively, may each utilize mutually exclusive moieties, chemistries, or structures.

For example, a nanoparticle may include a first polymer, the first polymer may include a hydrophilic, natural polyacrylamide functionalized with a plurality of azide side-chains. The nanoparticle may further include a second polymer, the second polymer may include a methacylate of small Mw and functionalized/pregrafted with a template site primer represented by Px, a primer used for orthogonal hybridization, which hybridizes to a portion of the template polynucleotide. The nanoparticle may further include a third polymer, the third polymer may include a lipophilic polymer, such as isopropylacrylamide, and functionalized with a plurality of amine side chains. The plurality of azide side-chains of the first polymer may be used to attach a first plurality of accessory oligonucleotides, wherein each oligonucleotide is functionalized with an alkyne group to facilitate azide-alkyne cycloaddition. The azide-alkyne cycloaddition may be Cu(I)-catalyzed (CuAAC) or Cu-free click (DBCO). The plurality of amine side chains of the third polymer may be used to attach a second plurality of accessory oligonucleotides, wherein each oligonucleotide is functionalized with an N-hydroxysuccinimide ester to facilitate NHC coupling. Accessory oligonucleotides may include primer P5 or primer P7, as described in greater detail below. In an implementation, each accessory oligonucleotide of the first plurality of accessory oligonucleotides is a read 1 primer P7 and each accessory oligonucleotide of the second plurality of accessory oligonucleotides is a read 2 primer P5. The activated patterned substrate, i.e., after masking and treatment to form wettable and non-wettable nanodomains, may then be dipped consecutively in the first polymer, second polymer, and third polymer to form a three-domain nanoparticle through entanglement and UV-triggered crosslinking. Thus, in this non-limiting example, the first polymer is dedicated to read 1, the third polymer is dedicated to read 2, and the second polymer is used for seeding and capturing an oligo of the template library. After seeding, the clustering step may be initiated by using a standard bridge Examp between the surface primers, P5 and P7, of the first and third polymers. Seeding, clustering, and paired-end read sequencing are described in greater detail below.

In an implementation, the first polymer and the third polymer are pre-functionalized with the first plurality of accessory nucleotides and the second plurality of accessory nucleotides, respectively.

A non-exclusive, non-limiting list of examples of complementary binding partners (e.g., moieties or structures) is presented in Table 1:

| | Example moiety/structure on (a) a | Example moiety/structure on (a) |
|---|---|---|
| | first, second, or third polymer or | template polynucleotide, template |
| | (b) template polynucleotide, | anchor oligonucleotide, or accessory |
| | template anchor oligonucleotide, | oligonucleotide or (b) the first, |
| Bonding site | or accessory oligonucleotide | second, or third polymer |
| amine-NHS | amine group, —NH₂ | N-Hydroxysuccinimide ester |
| | | 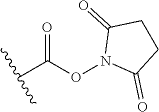 [SMILES: *C(=O)ON1C(=O)CCC1=O] |
| amine-imidoester | amine group, —NH₂ | imidoester |
| | | 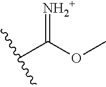 [SMILES: *C(=[NH2+])OC] |
| amine-pentofluorophenyl ester | amine group, —NH₂ | pentofluorophenyl ester, 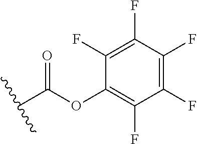 [SMILES: *C(=O)Oc1c(F)c(F)c(F)c(F)c1F] |
| amine-hydroxymethyl phosphine | amine group, —NH₂ | hydroxymethyl phosphine 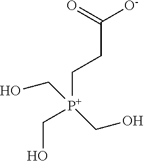 [SMILES: O=C([O-])CC[P+](CO)(CO)CO] |
| amine-carboxylic acid | amine group, —NH₂ | carboxylic acid group, —C(═O)OH |
| | | (e.g., following activation of the |
| | | carboxylic acid by a carbodiimide |
| | | such as EDC (1-ethyl-3-(-3- |
| | | dimethylaminopropyl)carbodiimide |
| | | hydrochloride) or DCC (N′,N′- |
| | | dicyclohexyl carbodiimide) to allow |
| | | for formation of an amide bond of |
| | | the activated carboxylic acid with an |
| | | amine group) |
| thiol-maleimide | thiol, —SH | maleimide |
| | | 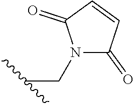 [SMILES: *CN1C(=O)C=CC1=O] |
| thiol-haloacetyl | thiol, —SH | haloacetyl (e.g., iodoacetyl or other |
| | | haloacetyl) |
| | | 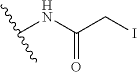 [SMILES: *NC(=O)CI] |
| thiol-pyridyl disulfide | thiol, —SH | pyridyl disulfide |
| | | 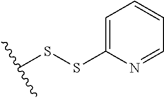 [SMILES: *SSc1ccccn1] |
| thiol-thiosulfonate | thiol, —SH | thiosulfonate |
| | | 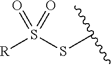 |
| thiol-vinyl sulfone | thiol, —SH | vinyl sulfone |
| | | 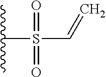 [SMILES: *S(=O)(=O)C=C] |
| aldehyde-hydrazide | aldehyde, —C(═O)H | hydrazide |
| | | 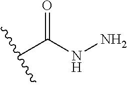 [SMILES: *C(=O)NN] |
| aldehyde-alkoxyamine | aldehyde, —C(═O)H | alkoxyamine |
| | | 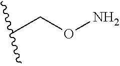 [SMILES: *CON] |
| hydroxy-isocyanate | hydroxyl, —OH | isocyanate |
| | | 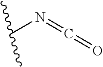 [SMILES: *N=C=O] |
| azide-alkyne | azide, —N₃ | alkyne |
| | | 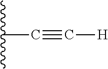 [SMILES: *C#C] |
| azide-phosphine | azide, —N₃ | phosphine, e.g.: |
| | | 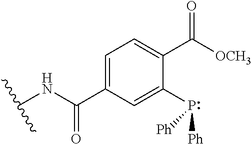 |
| azide-cyclooctyne | azide, —N₃ | cyclooctyne, e.g. dibenzocyclooctyne |
| | | 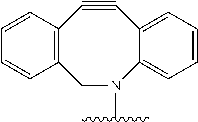 [SMILES: *N1Cc2ccccc2C#Cc2ccccc21] |
| | | or BCN (bicyclo[6.1.0]nonyne) |
| | | 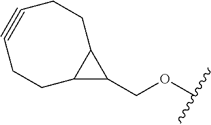 [SMILES: *OCC1C2CCC#CCCC21] |
| azide-norbornene | azine, —N₃ | norbornene |
| | | 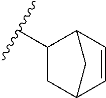 [SMILES: *C1CC2C=CC1C2] |
| transcyclooctene-tetrazine | transcyclooctene | tetrazine, e.g., benzyl- |
| | | methyltetrazine: |
| | 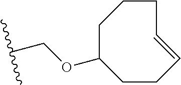 [SMILES: *COC1CC/C=C/CCC1] | 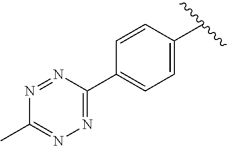 [SMILES: *c1ccc(-c2nnc(C)nn2)cc1] |
| norbornene-tetrazine | norbornene | tetrazine, e.g. benzyl-tetrazine |
| | 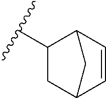 [SMILES: *C1CC2C=CC1C2] | 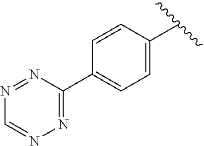 [SMILES: *c1ccc(-c2nncnn2)cc1] |
| oxime | aldehyde or ketone | alkoxyamine |

Any of the foregoing can be added to or included in the first, second, or third polymer as disclosed herein for attachment to a template polynucleotide or accessory oligonucleotide, which template polynucleotide or accessory oligonucleotide may include or be modified to include a complementary moiety or structure of the foregoing pairs for bonding. As a non-limiting example, accessory sites of a first polymer may include an azide moiety as a type of attachment site for a first plurality of accessory oligonucleotides. Correspondingly, the first plurality of accessory oligonucleotides may include a chemical moiety suitable for forming a covalent attachment to the azide attachment sites (e.g., alkyne, phosphine, cyclooctene, or norbornene sites). Or, the first plurality of accessory oligonucleotides may include azide attachment sites and the accessory attachment sites of the first polymer may include a chemical moiety suitable for forming a covalent attachment thereto (e.g., alkyne, phosphine, cyclooctene, or norbornene sites).

As another non-limiting example, accessory sites of a third polymer may include an amine moiety as a type of attachment site for a second plurality of accessory oligonucleotides. Correspondingly, the second plurality of accessory oligonucleotides may include a chemical moiety suitable for forming a covalent attachment to the amine attachment sites (e.g., NHS, imidoester, pentofluorophenyl ester, hydroxymethyl phosphine, or carboxylic acid sites). Or, the second plurality of accessory oligonucleotides may include amine attachment sites and the accessory attachment sites of the third polymer may include a chemical moiety suitable for forming a covalent attachment thereto (e.g., NHS, imidoester, pentofluorophenyl ester, hydroxymethyl phosphine, or carboxylic acid sites).

As would be appreciated by skilled persons, the foregoing examples could be reversed, with attachment sites of the first polymer comprising amine sites and the first accessory oligonucleotides comprising sites suitable for attachment thereto (e.g., NHS, imidoester, pentofluorophenyl ester, hydroxymethyl phosphine, or carboxylic acid sites), or vice versa, and the third polymer comprising azide sites and the second plurality of accessory oligonucleotides comprising sites suitable for attachment thereto (e.g., alkyne, phosphine, cyclooctene, or norbornene sites), or vice versa.

Similarly, accessory sites for a polymer (first polymer or third polymer) may include a thiol attachment site for a plurality of accessory oligonucleotides (first accessory oligonucleotides or second accessory oligonucleotides) and the corresponding accessory oligonucleotides (first or second) may include a moiety for forming a covalent attachment to the corresponding polymer, such as for example a maleimide, a haloacetyl, a pyridyl disulfide, a thiosulfonate, or vinyl sulfone), or vice versa. In another example, accessory sites for a polymer (first polymer or third polymer) may include an aldehyde attachment site for a plurality of accessory oligonucleotides (first accessory oligonucleotides or second accessory oligonucleotides) and the corresponding accessory oligonucleotides (first or second) may include a moiety for forming a covalent attachment to the corresponding polymer, such as a hydrazide or alkoxyamine, or vice versa. In another example, accessory sites for a polymer (first polymer or third polymer) may include a hydroxyl, transcyclooctene, norbornene, or aldehyde or ketone attachment site for a plurality of accessory oligonucleotides (first accessory oligonucleotides or second accessory oligonucleotides) and the corresponding accessory oligonucleotides (first or second) may include a moiety for forming a covalent attachment to the corresponding polymer, such as, respectively, an isocyanate, a tetrazine (e.g., a benzyl-methyltetrazine or benzyltetrazine), or an alkoxyamine, or vice versa, for forming a covalent attachment such as presented in Table 1.

The foregoing examples are all equally applicable to a single template site of a second polymer. For example, a second template site may include any one of the foregoing pairs of chemical moieties and an anchor oligonucleotide or template polynucleotide may include the other one of the foregoing pair of chemical moieties, and the moiety of the anchor oligonucleotide or template polynucleotide may form a covalent bod with the corresponding moiety of the second polymer, thereby forming a covalent bond between the template anchor oligonucleotide or template polynucleotide and the second polymer. A template anchor oligonucleotide may include a sequence of nucleotides that is complementary to a sequence of nucleotides of the template polynucleotide such that the template anchor oligonucleotide hybridizes to the template polynucleotide.

As skilled persons would understand, types of attachment sites for a first, second, and third polymer, and respective corresponding moieties on accessory oligonucleotides, template anchor oligonucleotides, or template polynucleotides for attachment thereto, may be chosen so as to selectively attach a first plurality of accessory oligonucleotides to a first polymer, a plurality of second accessory oligonucleotides to a third polymer, and a template anchor oligonucleotide or a template polynucleotide to the second polymer. That is, selecting from among different pairs of binding moieties, such as presented in Table 1 permits selection of mutually orthogonal binding chemistries, preventing, minimizing, or excluding, for example, binding of a template anchor oligonucleotide or template polynucleotide to a first or third polymer, first accessory oligonucleotides to a second or third polymer, second accessory oligonucleotides to a first or second polymer, or any combination of two or more of the foregoing.

Thus, any suitable bioconjugation method for adding or forming bonds between such pairs of complementary moieties or structures may be used. Modified nucleotides may be commercially available possessing examples of one or the other of examples of such pairs of complementary moieties or structures, and methods for including one or more of such examples of moieties or structures in or attaching or including them to polymer, a nucleotide, or polynucleotide are also known. Further diversification may be attained by the use of bifunctional linker molecules with a moiety or structure from one complementary pair of bonding partners listed in Table 1 at one end and a moiety or structure from another complementary pair of bonding partners listed in Table 1, connected by a polymer such as, as a nonlimiting example, polyethylene glycol or other polymer. A moiety or structure of a polymer, template polynucleotide, or of an accessory, or an oligo or polypeptide being attached to any of the foregoing features to as to provide a moiety or structure for bonding between any of such foregoing features, may be bound to one end of such a linker, resulting in the initial moiety or structure being effectively replaced with another, i.e., the moiety or structure present on the other end of the linker. Examples of suitable bioconjugation methods for adding or forming bonds between such pairs of complementary moieties or structures include bioconjugation methods as described in International Publication Number WO 2021/133768, which is hereby incorporated by reference in its entirety. Thus, for example, if a first polymer includes azide moieties as accessory sites, said accessory sites may be contacted with a bifunctional linker having at one end a moiety for covalently bonding to the azide moieties (e.g., alkyne, phosphine, cyclooctene, or norbornene sites) and at one end a different moiety for forming attachment with a plurality of first accessory oligonucleotides (e.g., thiol, maleimide, amide, or any other of the foregoing examples, without exception or limitation), thereby transforming the first type of attachment site of the first polymer from an azide to said other type of moiety on the other end of the bifunctional linker. The same could be accomplished by contacting an attachment site of a second polymer or an attachment site of a third polymer with a bifunctional linker, including all of the foregoing combinations and permutations, without exclusion or limitation.

In an implementation, the nanoparticle may undergo reorganization such that the shape of the nanoparticle is changed. Reorganization of the nanoparticle may occur at room temperature, upon cooling, or upon heating. For example, the first polymer and the third polymer include a first domain and a second domain, respectively, wherein the first domain is spatially separated from the second domain. Non-limiting examples of a first domain or a second domain include an N-Isopropylacrylamide (NIPAM), a polyacrylamide (PAM), and the like. In another implementation, the first domain and the second domain are NIPAM and PAM, respectively. It is understood that reorganization results in the particles having two distinct domains containing both the reverse and forward strands of the same clone. To undergo reorganization, the polymers should not be miscible, in other words, they should phase separate, e.g., the way oil and water do. Phase separation may be achieved by modifying the side chain of the polymer. In an example, a first polymer may be a hydrophilic polymer as disclosed herein and a third polymer may include the same or a different such hydrophilic polymer as disclosed herein, except that lipophilic chemical moieties may be substituted for side chains of said hydrophilic polymer, rendering the third polymer a lipophilic polymer. In another example, a third polymer may be a lipophilic polymer as disclosed herein and a first polymer may include the same or a different such lipophilic polymer as disclosed herein, except that hydrophilic chemical moieties may be substituted for side chains of said lipophilic polymer, rendering the first polymer a hydrophilic polymer. In another example, a first polymer and a third polymer may each include any given polymer backbone (irrespective of whether the polymer comprises a lipophilic polymer or hydrophilic polymer), whereas hydrophilic chemical moieties may be substituted for side chains of the first polymer rendering it a hydrophilic polymer and lipophilic chemical moieties may be substituted for side chains of the third polymer rendering it a lipophilic polymer.

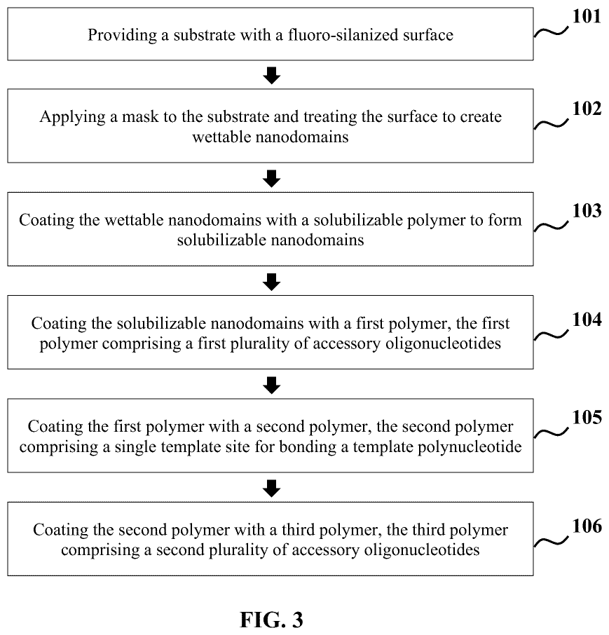

Another aspect relates to a method of making nanoparticles as disclosed herein. FIG. 3 depicts a flow diagram of a method of making such nanoparticles on the surface of a substrate. The method includes coating wettable nanodomains of a surface with a solubilizable polymer to form solubilizable nanodomains, coating the solubilizable nanodomains with a first polymer, coating the first polymer with a second polymer, coating the second polymer with a third polymer, and solubilizing the solubilizable polymer, wherein the first polymer includes a first plurality of accessory oligonucleotides, wherein the second polymer includes a single template site for bonding a template polynucleotide, and wherein the third layer includes a second plurality of accessory oligonucleotides.

Another aspect relates to a nanolithography-based approach to preparing a substrate on which to make the nanoparticles as disclosed herein. In an implementation, the substrate may be a silica-based substrate, such as glass, fused silica and other silica-containing materials. In another implementation, the silica-based substrate can also be silicon, silicon dioxide, silicon nitride, silicone hydrides. In yet another implementation, the substrate may include plastic materials such as polyethylene, polystyrene, poly(vinyl chloride), polypropylene, nylons, polyesters, polycarbonates and poly(methyl methacrylate). In still another implementation the substrate may be a metal. A non-limiting example of a metal substrate is gold. In a further implementation, the substrate surface includes a metal oxide. Non-limiting examples of metal oxide include tantalum oxide or titanium oxide.

Acrylamide, enone, or acrylate may also be utilized as a substrate material. Other substrate materials can include, but are not limited to gallium aresnide, indium phosphide, aluminum, ceramics, polyimide, quartz, resins, aryl azide, polymers and copolymers. The foregoing lists are intended to be illustrative of, but not limited to the present application.

In some embodiments, the substrate and/or the substrate surface can be quartz. In some other embodiments, the substrate and/or the substrate surface can be semiconductor, i.e., GaAs or ITO.

Substrates can include a single material or a plurality of different materials. Substrates can be composites or laminates. Substrate can be flat, round, textured and patterned. Patterns can be formed, for example, by metal pads that form features on non-metallic surfaces, for example, as described in U.S. patent application Ser. No. 13/661,524, which is incorporated herein by reference. Another useful patterned surface is one having well features formed on a surface, for example, as described in U.S. Ser. No. 13/787,396, US Pat. App. Pub. No. 2011/0172118 A1 or U.S. Pat. No. 7,622,294, each of which is incorporated herein by reference. For embodiments that use a patterned substrate, a gel can be selectively attached to the pattern features (e.g. gel can be attached to metal pads or gel can be attached to the interior of wells) or alternatively the gel can be uniformly attached across both the pattern features and the interstitial regions.

For the purposes herein, the term “masking” means a method of disposing a temporary protective layer over a region to physically prevent access. The term “unmasking” means the opposite in that is means a method of removing a protective layer to expose a region. The term “masked” means there is a temporary protective layer and the term “unmasked” means there is no protective layer.

In an implementation, a template may be used as a temporary mask to ensure that pre-defined nanodomains on a substrate are selectively treated to change their surface energy and thus their wettability in different solvent. For example, a template mask may be applied to a substrate such that some regions of the substrate are masked and some other regions are unmasked. The unmasked regions are then treated to change their surface energy. A non-limiting example of treatment to change surface energy includes exposing the unmasked regions to UV light and plasma ashing treatment. Non-limiting examples of masks that may be used include a photolithography mask, block copolymer lithography, or a nanoimprinted substrate with removable sacrificial resin interstitials.

In an implementation, the method includes coating wettable nanodomains of a surface with a solubilizable polymer to form solubilizable nanodomains, coating the solubilizable nanodomains with a first polymer, coating the first polymer with a second polymer, wherein the second layer includes a single template site for bonding a template polynucleotide coating the second polymer with a third polymer, and solubilizing the solubilizable polymer, attaching a first plurality of accessory oligonucleotides to the first layer, and attaching a second plurality of accessory oligonucleotides to the third layer.

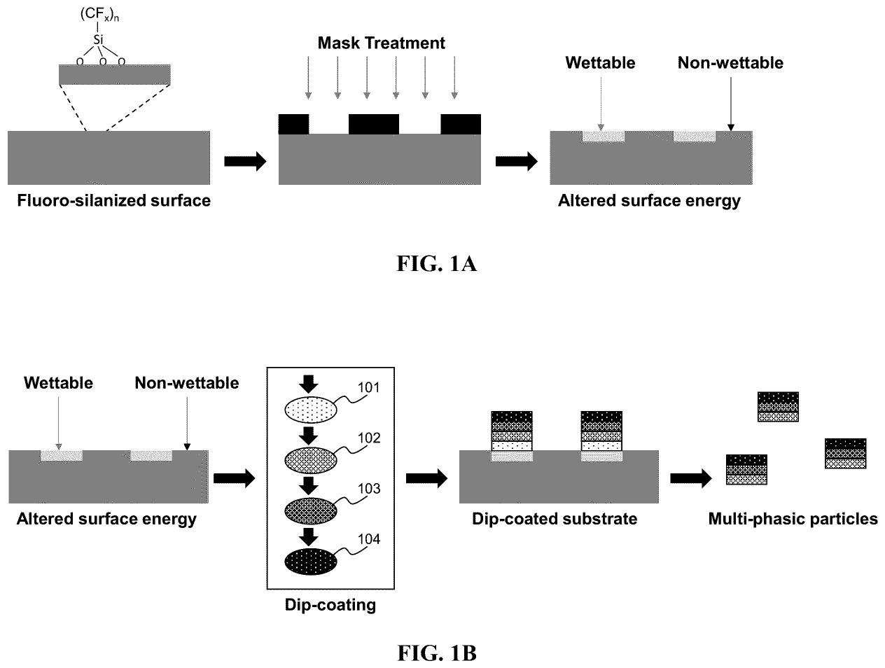

FIGS. 1A and 1B depict a method of making nanoparticles in accordance with aspects of the present disclosure. As depicted in FIG. 1A, in an implementation, application of a mask to a fluoro-silanized surface followed by a treatment process creates wettable and non-wettable nanodomains on the surface. Non-limiting examples of masks include photomasks, sacrificial patterned resins, and the like. For example, a photolithography mask, block copolymer lithography, or a nanoimprinted substrate with removable sacrificial resin interstitials may be used to mask the fluoro-silanized surface. Non-limiting examples of fluoro-silanized surfaces include those comprised of glass, Si, TaOₓ, or the like. Non-limiting examples of the treatment process include deep-UV, plasma, and the like. For example, exposure to UV light, especially at lower wavelengths, and plasma ashing treatment may be used to change the surface energy of the fluoro-silanized surface. In a non-limiting example, the wavelength of the UV light may be in the range of from about 100 to about 500 nm, including any and all subranges therein, for example from about 100 to about 370 nm, from about 250 to about 500 nm, from about 250 to about 370 nm, and the like. In addition, the wavelength may depend on the activation moiety. Portions of the fluoro-silanized surface that are covered by the mask become non-wettable interstices and portions of the fluoro-silanized surface that are exposed to the treatment process become wettable nanodomains.

As depicted in FIG. 1B, different polymers are sequentially dip-coated onto the wettable nanodomains. A solubilizable polymer 101 may be applied to the wettable domain, followed by a first polymer 102, a second polymer 103, and a third polymer 104. The solubilizable polymer may then be solubilized to yield nanoparticles with multiple functionalities. In non-limiting examples, the nanoparticles are multi-phasic particles comprised of functionalized polyacrylamide, or orthogonally reactive polymers, and the like.

Monomers used for formation of the first polymer 102 may include first accessory sites, in which the first accessory sites are attachment sites for a plurality of first oligonucleotides, or may include a plurality of first oligonucleotides. A plurality of first oligonucleotides may be attached to monomers of the first polymer 102 before polymerization of the first polymer 102, after polymerization of the first polymer 102 bue before solubilization of the solubilizable layer 101, or after solubilization of the solubilizable layer 101.

Monomers used for formation of the third polymer 104 may include second accessory sites, in which the second accessory sites are attachment sites for a plurality of second oligonucleotides, or may include a plurality of second oligonucleotides. A plurality of second oligonucleotides may be attached to monomers of the third polymer 104 before polymerization of the third polymer 104, after polymerization of the third polymer 104 but before solubilization of the solubilizable layer 101, or after solubilization of the solubilizable layer 101.

A second polymer 103 may include a single site for attachment of a template polynucleotide or a single site for attachment of a template anchor oligonucleotide, or may include a single template anchor oligonucleotide. A single site for attachment of a template polynucleotide, a single site for attachment of a template anchor oligonucleotide, or a single template anchor oligonucleotide may be added to the second polymer 103 during polymerization of the second polymer 103, after polymerization of the second polymer 103 but before the solubilizing, or after the solubilizing.

Lithographical methods enable a wide range, of from about 20 nm to about micrometers, of particle dimensions with regards to the horizontal template base, whereas the lateral dimension of the nanoparticles depends on the dip-coating parameters and may be on the order of from about 20 nm to about 2000 nm, including any and all subranges therein. In an implementation, the lateral dimension of the nanoparticles may be from about 100 to about 400 nm, including any and all subranges therein, for example from about 200 to about 250 nm, from about 200 to about 400 nm, from about 200 to about 350 nm, from about 200 to about 300 nm, from about 200 to about 300 nm, from about 100 to about 250 nm, or from about 150 to about 250 nm. In another implementation, the lateral dimension of the nanoparticles may be about 200, 210, 220, 230, 240, or 250 nm.

The geometry and composition of the nanoparticles may be precisely controlled using the methods described herein. Non-limiting examples of geometries of the nanoparticles include spheres, rods, cylinders, prisms of rectangles, triangles, or polygons, pyramids, disks, toroids, cones, and the like. In an implementation the nanoparticles may be configured such that they have disk-like shape. In another implementation, the nanoparticles may have a uniform shape. The shape of the nanoparticles may be determined by the wettable nanodomain formed on the fluoro-silanized surface.

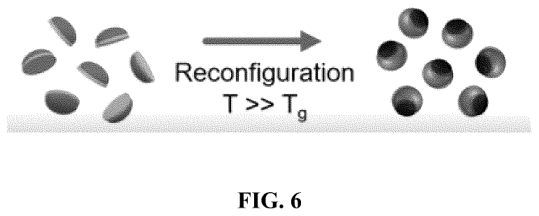

In yet another implementation, the shape of the nanoparticles may be reconfigured after the nanoparticle is formed. FIG. 6 illustrates the reconfiguration of nanoparticles as described herein via heating. In an implementation, a non-spherical nanoparticle may be heated to above the glass transition temperature of the polymers of which it is comprised, resulting in a spherical-shaped particle.

In an implementation, the polymer layers may be cross-linked by exposing the substrate to a suitable light source, such as 365 nm UV LED, after coating a formulation of the monomers mixed with a cross-linker or photoinitiator.

After the nanoparticles are released from the fluoro-silanized surface, the surface template may be re-used, making this method conducive to large-scale production of such nanoparticles. For example, the surface template may be re-used twice, five times, ten times, 20 times, 50 times, or more.

In an implementation, the substrate includes a nanowell. In another implementation, the substrate may be silanized TiO₂ or fluoro-silane TiO₂. In yet another implementation, attaching includes hybridizing a forward primer or a reverse primer to an oligonucleotide attached to the substrate.

In an implementation, one or both of attaching the first plurality of accessory oligonucleotides to the first layer and attaching the second plurality of accessory oligonucleotides to the third layer occurs before the solubilizing the solubilizable polymer. In another implementation, the solubilizable polymer may be a sulfonate, a sugar, or a phenol. In yet another implementation, the solubilizable polymer may be poly(sodium 4-styrenesulfonate) or poly-4-vinylphenol.

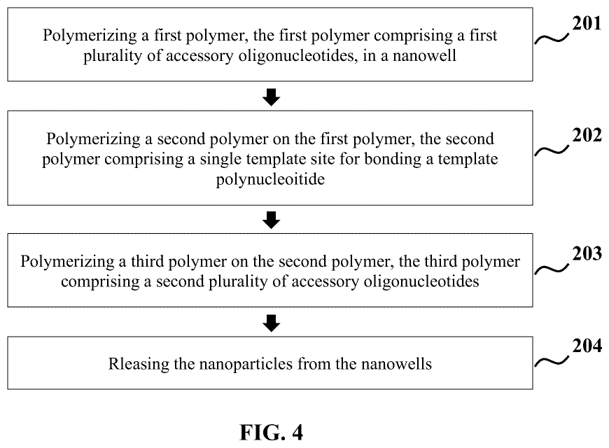

Another aspect relates to a method of making nanoparticles including forming the nanoparticles in nanowells and releasing the nanoparticles from the nanowells. As shown in FIG. 4, forming includes: polymerizing a first polymer including a first plurality of accessory oligonucleotides in a nanowell, polymerizing a second polymer including a single template site for bonding a template polynucleotide on the first polymer, and polymerizing a third polymer including a second plurality of accessory oligonucleotides on the second polymer. In an implementation, releasing the nanoparticles from the nanowells includes mechanically releasing the nanoparticles. Mechanically releasing the nanoparticles includes, for example, “squeezing out” the nanoparticles from the nanowells. In another implementation, the layer is flexible such that it can be rolled to release the particles.

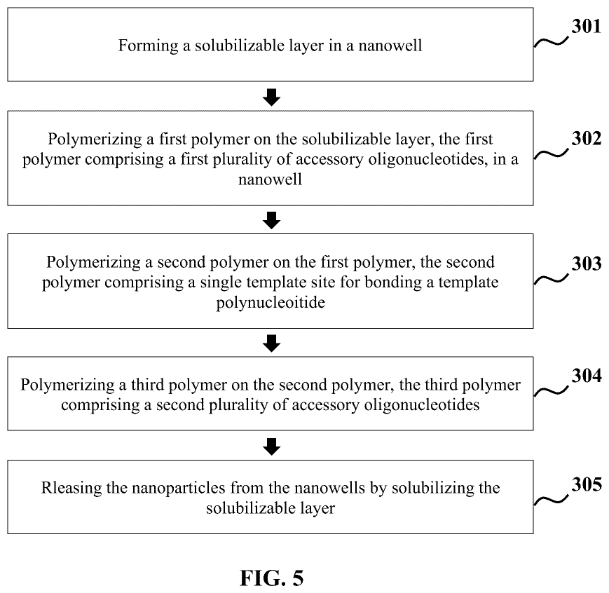

In another implementation, the method further includes forming a solubilizable layer in the nanowells before forming the nanoparticles in the nanowells. As shown in FIG. 5, a solubilizable polymer is polymerized in a nanowell before the first polymer is polymerized. In yet another implementation, releasing the nanoparticles from the nanowells includes solubilizing the solubilizable layer.

In a non-limiting example, a substrate surface is prepared for forming a nanoparticle as disclosed herein. When a smooth homogeneous surface (e.g., a substrate surface as described herein) contacts a liquid (e.g., a polymer solution as described herein), the liquid may wet the surface completely, or partially, making a finite equilibrium contact angle with the surface. The equilibrium contact angle (θ_{E}), given by Young's relation cos θ_{E}=(γ_{SV}−γ_{SL})/γ_{LV}, is determined by the balance between the solid-vapor (γ_{SV} or the surface energy), solid-liquid (γ_{SL}), and liquid-vapor (γ_{LV} or the surface tension) interfacial tensions acting at the three-phase contact line. The contact angle for a liquid as it advances from a smooth surface is called the advancing contact angle (θ_{A}) and the contact angle for a liquid as it recedes from a smooth surface is called the receding contact angle (θ_{R}). A substrate surface that is completely wettable has an θ_{R} equal to 0, and when it is pulled through a liquid (e.g., dip-coated) the substrate is coated with a liquid film of finite thickness, where the thickness is controlled by the velocity at which the substrate is pulled through the liquid. A substrate surface that is partially wettable has an θ_{R}>0, and when it is pulled through a liquid, the liquid film is unstable and dewets off the surface, leaving the surface dry for velocities below a certain value. Thus, when a patterned substrate surface with wettable nanodomains and non-wettable interstices between wettable nanodomains is pulled through a liquid, such as a liquid, only the wettable nanodomains remain coated by the liquid and the non-wettable interstices between wettable nanodomains remain uncoated.

In an example, a wettable nanodomain (or having high surface energy) may have, for both non-polar and polar liquids, a receding contact angle of less than or equal to about 5°, or less than or equal to about 4°, or less than or equal to about 3°, or less than or equal to about 2°, or less than or equal to about 10, or about 0°, reflecting a surface region that is wettable or substantially wettable by a variety of polar and non-polar substances applied thereto. In another example, a non-wettable interstice (or having low surface energy) between wettable nanodomains of a surface may have, for both non-polar and polar liquids, a receding contact angle of greater than or equal to about 10°, or greater than or equal to about 15°, or greater than or equal to about 20°, or greater than or equal to about 30°, or greater than or equal to about 40°, or greater than or equal to about 50°, or greater than or equal to about 60°, or greater than or equal to about 70°, or greater than or equal to about 80°, or greater than or equal to about 90°, reflecting a surface region that is non-wettable or substantially non-wettable by a variety of polar and non-polar substances applied thereto.

In a non-limiting example, a substrate with low surface energy, such as a fluoro-silanized surface or a titanium dioxide surface, may be masked (as described above) and the non-masked regions treated to alter the surface chemistry. The substrates with low surface energy possess finite receding contact angles for both aqueous and organic liquids and may be patterned with high surface energy, thereby creating wettable nanodomains. When the patterned surface is submerged and withdrawn from a polymer solution, the solution wets and self-assembles within the wettable nanodomains while receding from the non-wettable interstices, which have low surface energy. The feature size of the mask corresponds to the final lateral size of the nanoparticles. Thus, the non-masked, treated regions of the surface become wettable nanodomains while the masked regions remain non-wettable nanodomains. The mask may be removed from the substrate surface without affecting the surface energy modifications of the wettable nanodomains. The wettable nanodomains are coated with a solubilizable polymer, which may be solubilized after the nanoparticle is formed in order to release the nanoparticle from the substrate. In some implementations, the solubilizable polymer may be a sulfonate, a sugar, or a phenol. Non-limiting examples of solubilizable polymers include poly(sodium 4-styrenesulfonate) and poly-4-vinylphenol.

A first polymer including a first plurality of accessory oligonucleotides is coated onto the solubilizable polymer. The first plurality of accessory oligonucleotides is attached to the first polymer by a first chemistry or structure/moiety. A second polymer including a single template site for bonding a single template polynucleotide is coated onto the first polymer. A single template polynucleotide is attached to the single template site of the second polymer by a second chemistry or structure/moiety. A third polymer including a second plurality of accessory oligonucleotides is coated onto the second polymer. The second plurality of accessory oligonucleotides is attached to the third polymer by a third chemistry or structure/moiety. The attachment of the first plurality of accessory nucleotides, second plurality of accessory nucleotides, and template polynucleotide is orthogonal, in that each of the first, second, and third chemistries or structures/moieties are different from each other and do not cross-react. The means of attachment are described in Table 1, above.

The nanoparticle may be released from the substrate surface by solubilizing the solubilizable polymer. The nanoparticle may be used in SBS sequencing systems, as described in greater detail below.

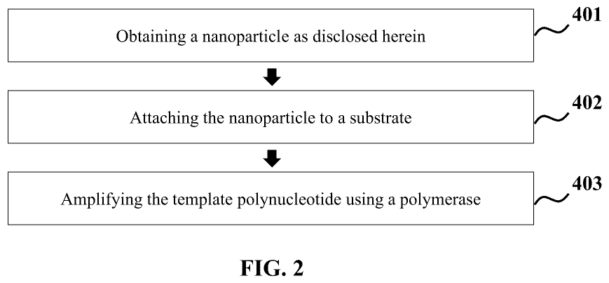

Another aspect relates to a method of amplifying a template polynucleotide, as shown in FIG. 2. The method includes attaching a nanoparticle disclosed herein to a substrate and amplifying the template polynucleotide using a polymerase. In an implementation, the substrate includes a nanowell. In another implementation, the substrate may be silanized TiO₂ or fluoro-silane TiO₂. Non-limiting examples of suitable substrates include glass, NIL resin, laminate, TaOx, and the like. The substrates may be patterned or continuous. In a non-limiting example, the substrate may be NIL with a patterned surface and a 200-250 nm feature size. In still another implementation, attaching includes hybridizing a forward primer or a reverse primer to an oligonucleotide attached to the substrate.

Also, it is to be understood that amplification and/or sequencing of polynucleotide strands (e.g., forward strands or reverse strands) may not always produce exact duplicates of the strands or exact duplicates of the reverse complements of the strands. This is because, for various factors, errors may be introduced in amplification and/or sequencing process, which may introduce defects (e.g., the incorrect base) in the polynucleotide sequence of bases. For example, there may be up to 1 out of a million defects, 10 out of a million defects or 100 out of a million defects introduced into the sequenced or amplified strands. Accordingly, a cluster of forward or reverse strands 121, 123 may not contain exact duplicates of each strand in the cluster but may include substantially the same duplicates of each strand in the cluster.

Cluster amplification is an approach to amplifying polynucleotides, for example for use in genetic sequencing. Target polynucleotides are captured by primers (e.g., P5 and P7 primers) coupled to a substrate surface in a flowcell, and form “seeds” at random locations on the surface. Cycles of amplification may be performed to form clusters on the surface around each seed. The clusters include copies, and complementary copies, of the seed polynucleotides. In some circumstances, the substrate may be patterned so as to define regions that bound different clusters, such as wells that may be filled with respective clusters.

A variety of amplification techniques may be used, including, but not limited to, polymerase chain reaction (PCR), rolling circle amplification (RCA), multiple displacement amplification (MDA), or random prime amplification (RPA). In some examples the amplification can be carried out in solution, for example, when the amplification sites are capable of containing amplicons in a volume having a desired capacity. Preferably, an amplification technique used under conditions of kinetic exclusion in a method of the present disclosure will be carried out on solid phase. For example, one or more primers used for amplification can be attached to a solid phase at the amplification site. In PCR examples, one or both of the primers used for amplification can be attached to a solid phase. Formats that utilize two species of primer attached to the surface are often referred to as bridge amplification because double stranded amplicons form a bridge-like structure between the two surface-attached primers that flank the template sequence that has been copied. Example reagents and conditions that can be used for bridge amplification are described, for example, in U.S. Pat. No. 5,641,658; U.S. Patent Publ. No. 2002/0055100; U.S. Pat. No. 7,115,400; U.S. Patent Publ. No. 2004/0096853; U.S. Patent Publ. No. 2004/0002090; U.S. Patent Publ. No. 2007/0128624; and U.S. Patent Publ. No. 2008/0009420, each of which is incorporated herein by reference in its entirety.

In an implementation, the principles of size exclusion are used to prevent individual template polynucleotides from seeding too close to each other and thereby promoting adjoined/comingled clusters. By associating each individual template polynucleotide with a nanoparticle of a given, sufficient spatial dimension, the template polynucleotides may be induced to attach to a substrate's surface sufficiently distal from each other to reduce formation of polyclonal clusters and increase formation of monoclonal clusters. In another implementation, the second polymer includes a single template site for bonding a template polynucleotide. The second polymer may have only one, single site for attachment of a template polynucleotide. One and only one template polynucleotide may therefore be capable of attaching to the nanoparticle, such that attachment of a template polynucleotide to the second polymer prevents attachment of a second template polynucleotide to the same nanoparticle, the attached template polynucleotide having occupied the single template polynucleotide bonding site thereof. Attachment of only a single template polynucleotide per nanoparticle and resulting spatial distribution of template polynucleotides attached to such nanoparticles from each other due, directly or indirectly, to the sizes of the attached nanoparticles, reduces formation of polyclonal clusters.

In an example of preparation of polynucleotide strands for a sequencing process, a first adapter and a different second adapter are often added to the ends of the polynucleotide strands, to form what is known as a DNA library. The adapters are complementary to forward and reverse primers, such as oligonucleotide fragments (oligos), which are anchored in the nanowells of the flow cell by their 5′ ends. Thus, the DNA library to be sequenced thus hybridizes (seeds) to the forward and reverse primers and may be amplified on the solid support forming a DNA cluster.

The forward and reverse primer contain chemical cleavage sites, such that the forward strands or reverse strands may be cleaved and removed independently. Sequencing of the forward and reverse strands may be carried out in a sequential manner, by first removing reverse strands, blocking their 3′ ends, and sequencing the forward strands resulting in a read 1, and then after the cluster has been reamplified, the forward strands are removed, their 3′ ends blocked, and sequencing the reverse strands, resulting in a read 2.

Because, in this example, synthesis of forward strands and reverse strands are done serially, the process may be very time consuming. Additionally, the larger the nanowells (for example for larger clusters or multiple clusters), the more the probability that polyclonality (i.e., more than one type of strand being initially seeded in the nanowell and then simultaneously amplified into a polyclonal cluster) may occur. Moreover, the closer clusters are to each other, the more the probability of crosstalk (i.e., light emitted from one cluster entering the light guide of another cluster and registering on an unassociated light detector) may occur.

Simultaneous paired-end sequencing allows users to sequence both forward and reverse complementary strands of a cluster at the same time. Additionally, nanoparticles of the present disclosure enable a reduced probability of polyclonality and crosstalk for adjacent forward and reverse strand clusters and physical separation of forward and reverse reads to permit simultaneous sequencing reads thereof. Examples of a method for simultaneous paired-end sequencing of template polynucleotides is disclosed in, for example, U.S. Pat. No. 11,124,824, which is incorporated herein by reference in its entirety.

Paired end sequencing involves 2 reads from the two ends of a fragment. Paired end reads are used to resolve ambiguous alignments. Paired-end sequencing allows users to choose the length of the insert (or the fragment to be sequenced) and sequence either end of the insert, generating high-quality, alignable sequence data. Because the distance between each paired read is known, alignment algorithms can use this information to map reads over repetitive regions more precisely. This results in better alignment of the reads, especially across difficult-to-sequence, repetitive regions of the genome. Paired-end sequencing can detect rearrangements, including insertions and deletions (indels) and inversions. Methods for fragmenting a target nucleic acid sample (e.g., genomic DNA sample), attaching primers to accommodate paired end reads and reading sequence from the ends of the fragments are known and can be carried out as described, for example, in U.S. Pat. Nos. 7,754,429; 8,017,335; and 8,192,930, each of which is incorporated herein by reference.

In paired-end read sequencing, each of the first primer sets includes an un-cleavable first primer and a cleavable second primer, and each of the second primer sets includes a cleavable first primer and an un-cleavable second primer. The un-cleavable first primer and the cleavable second primer are oligo pairs, where the un-cleavable first primer is a forward amplification primer and the cleavable second primer is a reverse amplification primer, or the cleavable second primer is the forward amplification primer and the un-cleavable first primer is a reverse amplification primer. In each example of the first primer set, the cleavable second primer includes a cleavage site while the un-cleavable first primer does not include a cleavage site.

The cleavable first primer and the un-cleavable second primer are also oligo pairs, e.g., where the cleavable first primer is a forward amplification primer and un-cleavable second primer is a reverse amplification primer, or where the un-cleavable second primer is a forward amplification primer and the cleavable first primer is a reverse amplification primer. It is to be understood that the un-cleavable first primer of the first primer set and the cleavable first primer of the second primer set have the same nucleotide sequence (e.g., both are forward amplification primers, or both are reverse amplification primers), except that the cleavable primer has a cleavage site integrated into the nucleotide sequence or into a linker attached to the nucleotide sequence. It is to be understood that when the first primers are forward amplification primers, the second primers are reverse primers, and vice versa. Examples of un-cleavable primers include P5 and P7 primers, examples of which are used on the surface of commercial flow cells sold by Illumina Inc. for sequencing, for example, on HISEQ™, HISEQX™, MISEQ™, MISEQDX™ MINISEQ™, NEXTSEQ™, NEXTSEQDX™, NOVASEQ™, ISEQ™, GENOME ANALYZER™, and other instrument platforms. The P5 and P7 primers have a universal sequence for capture and/or amplification purposes.

In an example, a nanoparticle as disclosed herein may be used in simultaneous paired-end sequencing. Orthogonal chemistries for binding of a first type of primers on one face of a nanoparticle and a second type of primers on an opposing face of a nanoparticle may promote physical separation of copies of a template polynucleotide and its complement on opposing faces following clustering. For example, two faces of a nanoparticle may include forward and reverse primers for clustering amplification of a template polynucleotide, with only a single site for attachment for a template polynucleotide included in a layer between those of the opposing faces. On one face, forward primers may be cleavable from the nanoparticle (such as by including a modified nucleotide such as a modified uracil) and reverse primers non-cleavable, and on the opposing face reverse primers may be cleavable and forward primers non-cleavable, or vice versa. For example, on one face, the un-cleavable first primer may be P7 and the cleavable, e.g., uracil-modified, second primer may be P5U; and on the other face, the cleavable, e.g., uracil-modified, first primer may be P7U and the un-cleavable second primer may be P5. As described above, the chemistry of the first and second primers is orthogonal, which allows for amplification across both sets, e.g., P7/P5U and P7U/P5, and cleavage of some of the generated template strands, leaving the same (forward or reverse) template strands in a particular region. This enables distinguishable read 1 and read 2 signals to be obtained simultaneously. Following amplification, amplicons attached to the nanoparticle by cleavable primers may be cleaved from the nanoparticle, leaving only forward amplicons attached to one face of the nanoparticle and reverse amplicons attached to the opposing face of the nanoparticle, or vice versa. Simultaneous reads from forward and reverse strands may thereby be facilitated by reduction of physical overlap of fluorescent signal emitted from amplicons attached to each opposing face (e.g., physical separation of emitted fluorescent signal).

The use, if any, of ordinal indicators, e.g., (a), (b), (c) . . . or the like, in this disclosure and claims is to be understood as not conveying any particular order or sequence, except to the extent that such an order or sequence is explicitly indicated. For example, if there are three steps labeled (i), (ii), and (iii), it is to be understood that these steps may be performed in any order (or even concurrently, if not otherwise contraindicated) unless indicated otherwise. For example, if step (ii) involves the handling of an element that is created in step (i), then step (ii) may be viewed as happening at some point after step (i). Similarly, if step (i) involves the handling of an element that is created in step (ii), the reverse is to be understood.

It is to be appreciated that certain aspects, modes, implementations, variations, and features of the present disclosure are described below in various levels of detail in order to provide a substantial understanding of the present technology. Unless otherwise noted, all technical and scientific terms used herein generally have the same meaning as commonly understood by one of ordinary skill in the art. The use of the term “including” as well as other forms is not limiting. The use of the term “having” as well as other forms is not limiting. As used in this disclosure, whether in a transitional phrase or in the body of the claim, the terms “comprise(s)” and “comprising” are to be interpreted as having an open-ended meaning. That is, the terms are to be interpreted synonymously with the phrases “having at least” or “including at least.”

It is also to be understood that the use of “to,” e.g., “a valve to switch between two flow paths,” may be replaceable with language such as “configured to,” e.g., “a valve configured to switch between two flow paths”, or the like.

The terms “substantially”, “approximately”, “about”, “relatively”, or other such similar terms that may be used throughout this disclosure, including the claims, are used to describe and account for small fluctuations, such as due to variations in processing, from a reference or parameter. Such small fluctuations include a zero fluctuation from the reference or parameter as well. For example, fluctuations can refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

It is further appreciated that certain features described herein, which are, for clarity, described in the context of separate implementations, can also be provided in combination in a single implementation. Conversely, various features which are, for brevity, described in the context of a single implementation, can also be provided separately or in any suitable sub-combination.

In the present disclosure, reference is made to the accompanying drawings that form a part hereof, and in which is shown by way of illustration specific implementations which may be practiced. These implementations are described in detail to enable those skilled in the art to practice the disclosure, and it is to be understood that other implementations may be utilized and that structural, logical and electrical changes may be made without departing from the scope of the present disclosure.

The terms “connect”, “contact”, and/or “coupled” include a variety of arrangements and assemblies. These arrangements and techniques include, but are not limited to, (1) the direct joining of one component and another component with no intervening components therebetween (i.e., the components are in direct physical contact); and (2) the joining of one component and another component with one or more components therebetween, provided that the one component being “connected to” or “contacting” or “coupled to” the other component is somehow in operative communication (e.g., electrically, fluidly, physically, optically, etc.) with the other component (optionally with the presence of one or additional components therebetween). Components that are in direct physical contact with one another may or may not be in electrical contact and/or fluid contact with one another. Moreover, two components that are electrically connected, electrically coupled, optically connected, optically coupled, fluidly connected, or fluidly coupled may or may not be in direct physical contact, and one or more other components may be positioned between those two connected components.

### Examples

The following examples are intended to illustrate, but by no means are intended to limit, the scope of the present disclosure as set forth in the appended claims.

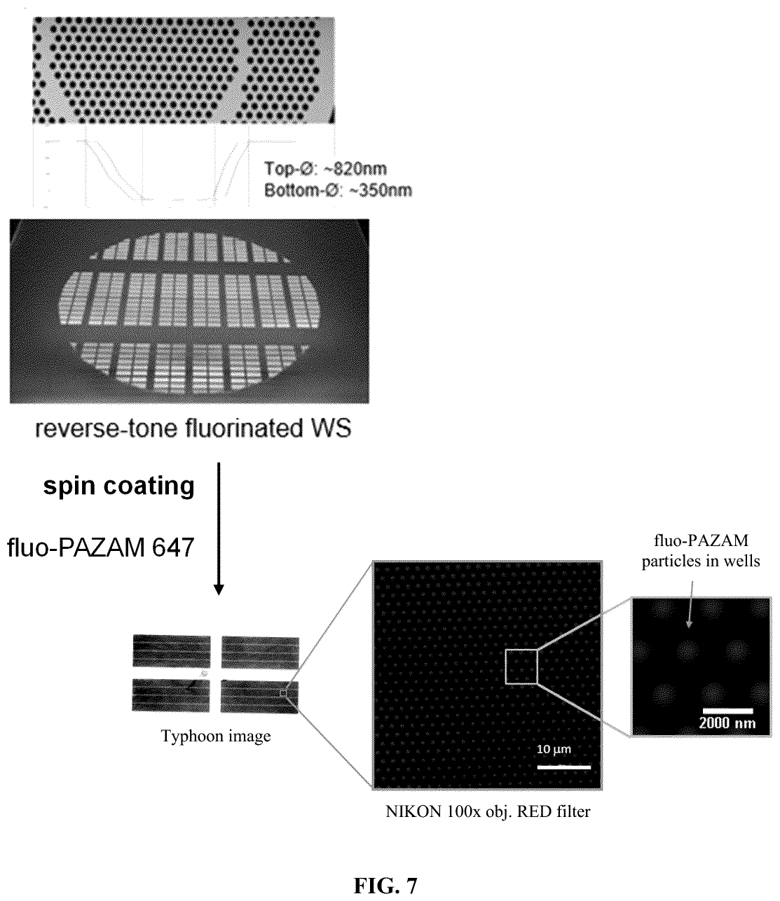

FIG. 7 is a workflow scheme depicting the making and detecting of polymer particles made with a nanoimprint lithography working stamp.

A reverse-tone fluorinated working stamp was filled with fluorescently tagged azapa-co-acrylamide (PAZAM) polymer by spin-coating the aqueous solution of the latter onto it (0.25 weight % with 5% ethanol added). The working stamp was gently wiped to remove any interstitial polymer, and the remaining material in the nanowells was then cured at 60° C. for 1 hour. The fluorescent confocal image of the working stamp below shows that the physical barriers imposed by the working stamp feature facilitate the polymer particles to be segregated by gravity into the desired templated nanofeatures.

Although preferred implementation have been depicted and described in detail herein, it will be apparent to those skilled in the relevant art that various modifications, additions, substitutions, and the like can be made without departing from the spirit of the subject matter disclosed herein and these are therefore considered to be within the scope of subject matter as defined in the claims which follow.

1. A nanoparticle comprising:
a first layer comprising a first polymer and a first plurality of accessory sites,
a second layer comprising a second polymer comprising a single template site for bonding a template polynucleotide, and
a third layer comprising a third polymer and a second plurality of accessory sites,
wherein the second layer is between the first layer and the third layer.

2. The nanoparticle of claim 1, wherein one or both of:
(i) the first polymer is a hydrophilic polymer; and
(ii) the third polymer is a lipophilic polymer.

3. The nanoparticle of claim 2, wherein one or both of:
(i) the hydrophilic polymer is selected from a natural polyacrylamide, a polyethylene imine, a polypeptide, a polysaccharide, a polyvinyl alcohol, a poly acrylic acid, a poly allylamine, a poly-styrene sulfonate, or a poly-oxazoline; and
(ii) the lipophilic polymer is selected from an isopropylacrylamide, an acrylic, an epoxy, a polyethylene, a polystyrene, a polyvinyl a polymethyl sulfonate, a polyurethane, and a fluorinated polymer.

4-5. (canceled)

6. The nanoparticle of claim 1, wherein the first polymer and the third polymer are each independently chosen from a poly(vinylidene fluoride), a polystyrene, an epoxy polymer, a (meth)acrylate polymer, a polydimethylsiloxane, an SiO₂-containing polymer, a poly(lactic-co-glycolic acid) polymer, a perfluorinated polymer, an azapa-co-acrylamide polymer (PAZNAM), a poly(N-(5-azidoacetamidylpentyl)acrylamide-co-acrylamide) (PAZAM) polymer, a poly(o-nitrobenzyl-masked acrylamide-co-acrylamide) copolymer, a poly(benzopyrone-masked acrylamide-co-acrylamide) copolymer, a poly(aminotriazole-acrylamide-co-acrylamide) copolymer, a poly(thiotriazole-acrylamide-co-acrylamide) copolymer, a poly(alkenyltriazole-acrylamide-co-acrylamide) copolymer, and a thiol/ene cross-linkable monomer mix.

7. The nanoparticle of claim 1, wherein one or more of:
(i) the first polymer is polyacrylamide;
(i) the third polymer is isopropyl acrylamide; and
(ii) the second polymer is methacylate.

8. The nanoparticle of claim 1, wherein the second polymer is a copolymer of the first polymer and the third polymer.

9-10. (canceled)

11. The nanoparticle of claim 1, further comprising one or both of a first plurality of accessory oligonucleotides attached to the first plurality of accessory sites and a second plurality of accessory oligonucleotides attached to the second plurality of accessory sites.

12. The nanoparticle of claim 11, wherein one or both of the first plurality of accessory oligonucleotides and the second plurality of accessory oligonucleotides comprise one or both of forward primers and reverse primers, wherein sequences of the forward primers and sequences of the reverse primers permit amplifying the template polynucleotide by a polymerase.

13. The nanoparticle of claim 12, wherein forward primers of the first plurality of accessory oligonucleotides and reverse primers of the second plurality of accessory oligonucleotides, or reverse primers of the first plurality of accessory oligonucleotides and forward primers of the second plurality of accessory oligonucleotides, are cleavable, and other primers of the first and second pluralities of accessory oligonucleotides are uncleavable.

14. The nanoparticle of claim 1, wherein the single template site comprises:
a type of attachment site for a template anchor oligonucleotide or the template anchor oligonucleotide.

15. A method comprising:
attaching the nanoparticle of claim 1 to a substrate and amplifying the template polynucleotide using a polymerase.

16-17. (canceled)

18. The method of claim 15, wherein one or both of:
(i) the substrate is silanized TiO₂ or fluoro-silane TiO₂; and
(ii) attaching comprises hybridizing a forward primer or a reverse primer to an oligonucleotide attached to the substrate.

19. A method of making a nanoparticle comprising:
coating wettable nanodomains of a substrate surface with a solubilizable polymer, wherein the wettable nanodomains are separated by non-wettable interstices;
coating the solubilizable polymer with a first polymer, wherein the first polymer comprises a first plurality of accessory sites;
coating the first polymer with a second polymer, wherein the second polymer comprises a single template site;
coating the second polymer with a third polymer, wherein the third polymer comprises a second plurality of accessory sites; and
solubilizing the solubilizable polymer to release the nanoparticle.

20. The method of claim 19, wherein one or both of:
(i) the first plurality of accessory sites comprises a first plurality of accessory oligonucleotides or the first plurality of accessory sites comprises a first type of attachment site for the first plurality of accessory oligonucleotides but not for a second plurality of accessory oligonucleotides;
(ii) the second plurality of accessory sites comprises a second plurality of accessory oligonucleotides or the second plurality of accessory sites comprises a second type of attachment site for the second plurality of accessory oligonucleotides but not for the first plurality of accessory oligonucleotides; and
(iii) the single template site comprises:
a type of attachment site for a template anchor oligonucleotide and the method further comprises attaching the template anchor oligonucleotide to the single template site; or
the template anchor oligonucleotide.

21. (canceled)

22. The method of claim 20, wherein one or both of:
(i) attaching the first plurality of accessory oligonucleotides to the first plurality of accessory sites occurs before coating the solubilizable polymer with the first polymer or after coating the solubilizable polymer with the first polymer and before the solubilizing, or after the solubilizing; and
(ii) attaching the second plurality of accessory oligonucleotides to the second plurality of accessory sites occurs before coating the solubilizable polymer with the first polymer or after coating the solubilizable polymer with the first polymer and before the solubilizing, or after the solubilizing.

23. (canceled)

24. The method of claim 19, wherein the solubilizable polymer is a sulfonate, a sugar, or a phenol.

25. The method of claim 24, wherein the solubilizable polymer is poly(sodium 4-styrenesulfonate) or poly-4-vinylphenol.

26. A method comprising:
forming a nanoparticle in a nanowell, wherein forming comprises:
polymerizing a first polymer, wherein the first polymer comprises a first plurality of accessory sites;
polymerizing a second polymer on the first polymer, wherein the second polymer comprises a single template site; and
polymerizing a third polymer on the second polymer, wherein the third polymer comprises a second plurality of accessory sites.

27. The method of claim 26, wherein one or more of:
(i) the first plurality of accessory sites comprises a first plurality of accessory oligonucleotides or the first plurality of accessory sites comprises a first type of attachment site for the first plurality of accessory oligonucleotides but not for a second plurality of accessory oligonucleotides;
(ii) the second plurality of accessory sites comprises a second plurality of accessory oligonucleotides or the second plurality of accessory sites comprises a second type of attachment site for the second plurality of accessory oligonucleotides but not for the first plurality of accessory oligonucleotides; and
(iii) the single template site comprises:
type of attachment site for a template anchor oligonucleotide and the method further comprises attaching the template anchor oligonucleotide to the single template site; or
the template anchor oligonucleotide.

28-29. (canceled)

30. The method of claim 26, further comprising one of:
(i) releasing the nanoparticles from the nanowells, wherein releasing comprises mechanically releasing the nanoparticles; and
(ii) polymerizing a solubilizable polymer before the polymerizing a first polymer and releasing the nanoparticles from the nanowells, wherein releasing comprises solubilizing the solubilizable polymer.

31-59. (canceled)
